(19) United States
(12) Patent Application Publication
(10) Pub. No.: US 2024/0181128 A1
(43) Pub. Date: Jun. 6, 2024

(54) BIORESORBABLE IMPLANT MATERIALS AND METHODS OF MAKING THE SAME

(71) Applicant: Georgia Tech Research Corporation, Atlanta, GA (US)

(72) Inventors: Sriharsha Ramaraju, Atlanta, GA (US); Scott J. Hollister, Atlanta, GA (US)

(21) Appl. No.: 18/287,475

(22) PCT Filed: May 19, 2022
(86) PCT No.: PCT/US2022/072442

§ 371 Date: Oct. 19, 2023

Related U.S. Application Data
(60) Provisional application No. 63/190,434, filed on May 19, 2021.

(51) Int. Cl. A61L 27/18; A61L 27/56; A61L 27/58; C08L 67/02

(52) U.S. Cl. CPC A61L 27/18; A61L 27/56; A61L 27/58; C08L 67/02; A61L 2300/604; A61L 2400/16; C08L 2201/06; C08L 2201/12

(57) ABSTRACT

Disclosed herein are implant materials comprising a shape memory polymer having a first shape and a second shape. The shape memory polymer can comprise at least one monomer unit of glycerol and at least one monomer unit of dodecanedioate and a functionalized surface. The shape memory polymer can take the first shape at a first environmental temperature and the second shape at a second environmental temperature, the second environmental temperature being greater than the first environmental temperature. Also disclosed herein are methods of making the same.

### CROSS-REFERENCE TO RELATED APPLICATIONS

This application claims the benefit of U.S. Provisional Application Ser. No. 63/190,434, filed on 19 May 2021, the entire contents and substance of which is incorporated herein by reference in its entirety as if fully set forth below.

### STATEMENT OF RIGHTS UNDER FEDERALLY SPONSORED RESEARCH

This invention was made with government support under Grant No. 21NL126004 awarded by National Institute for Health and Care Research. The government has certain rights in the invention.

### FIELD OF THE DISCLOSURE

The present disclosure relates generally to bioresorbable implant materials and methods. Particularly, embodiments of the present disclosure relate to bioresorbable implant materials formed from shape memory polymers and methods of making the same.

### BACKGROUND

Cardiovascular diseases are rising globally due to an aging and growing population. Acquired heart disease with associated sequelae and congenital heart defects are associated with high mortality rates, associated with high hospitalization rates, and incur the largest healthcare utilization costs within United States. Congenital heart disease (CHD) has a high incidence rate (0.4-1 in 100 live births) and is the leading cause of hospitalization due to congenital abnormalities exhibiting high comorbidities and rates of surgical revision procedures. Although surgical and medical advances reduce mortality rates, the prevalence of individuals living with CHDs has increased with a higher number of adults surviving sequelae.

Minimally invasive percutaneous interventions for cardiovascular diseases are increasingly preferred due to reduced resource utilization and fewer adverse events compared to open procedures. A number of percutaneous procedures have gained prominence in clinical practice including but not limited to endovascular stenting, valve repair and replacement, and closure of atrial septal defects (ASD) and patent foramen ovale (PFO) and patent ductus arteriosus. In nearly all of these minimally invasive procedures, devices seen as a gold standard utilize a metallic alloy framework (e.g., Nickel-titanium or cobalt-chromium) often times delivered with a biocompatible or bioactive polymer patch, coating, or extracellular matrix material to promote endothelialization and/or limit erosion and tissue damage from the metallic framework. Polymers used in conjunction with these materials can reduce acute and late-stage adverse consequences. For instance, polymer coatings and drug-eluting polymer coated metal stents account for over 80% of stent use because they reduce inflammation and restenosis.

Additionally, metal prosthetic implants are increasingly expected to perform throughout the lifetime of the patient which remains an exceptionally high hurdle for pediatric interventions due to tissue growth. Consequently, tissue engineering and regenerative medicine approaches to heal cardiovascular pathologies are of increasing clinical interest to address these needs of aging and growing populations.

What is needed, therefore, are bioresorbable implant materials and methods of making the same. Embodiments of the present disclosure address this need as well as other needs that will become apparent upon reading the description below in conjunction with the drawings.

### BRIEF SUMMARY OF THE DISCLOSURE

The present disclosure relates generally to bioresorbable implant materials and methods. Particularly, embodiments of the present disclosure relate to bioresorbable implant materials formed from shape memory polymers and methods of making the same.

An exemplary embodiment of the present disclosure can provide an implant material comprising: a shape memory polymer having a first shape and a second shape, the shape memory polymer comprising: at least one monomer unit of glycerol and at least one monomer unit of dodecanedioate; and a functionalized surface, wherein the shape memory polymer takes the first shape at a first environmental temperature and the second shape at a second environmental temperature, the second environmental temperature being greater than the first environmental temperature.

In any of the embodiments disclosed herein, the shape memory polymer can have a melt transition temperature from approximately 25° C. to approximately 45° C., and the melt transition temperature is greater than or equal to the first environmental temperature and less than the second environmental temperature.

In any of the embodiments disclosed herein, the melt transition temperature can be from approximately 31° C. to 35° C.

In any of the embodiments disclosed herein, the shape memory polymer can be an elastomer above the melt transition temperature a thermoplastic in the below the melt transition temperature.

In any of the embodiments disclosed herein, the first shape can be a curved or tubular shape.

In any of the embodiments disclosed herein, the second shape can be a tubular shape comprising a cut along a longitudinal axis of the implant material.

In any of the embodiments disclosed herein, the functionalized surface can comprise a plurality of suture holes laser cut into the functionalized surface.

In any of the embodiments disclosed herein, the functionalized surface can comprise at least one functional group bonded to the shape memory polymer.

In any of the embodiments disclosed herein, the at least one functional group can comprise a bioactive agent.

In any of the embodiments disclosed herein, the molar ratio of the at least one monomer unit of glycerol to the at least one monomer unit of dodecanedioate can be from approximately 10:1 to approximately 1:10.

In any of the embodiments disclosed herein, the implant material can have a biodegradation time when implanted in vivo from approximately 4 months to approximately 24 months.

Another embodiment of the present disclosure can provide a method of making an implant, the method comprising forming the implant material of any of the embodiments disclosed herein into one or more of an implant, a patterned mesh, or a molded medical device.

In any of the embodiments disclosed herein, the implant can be in the form of a sheet, a membrane, a mesh, a sponge, a patch, a molded medical device, or a combination thereof.

Another embodiment of the present disclosure can provide a method of making an implant material, the method comprising: depositing a shape memory polymer resin to a mold, the shape polymer resin comprising at least one monomer unit of glycerol and at least one monomer unit of dodecanedioate; partially curing the shape memory polymer resin to form a shape memory elastomer; cutting the shape memory elastomer; and curing the shape memory elastomer to form the implant material, wherein the shape memory elastomer takes a first shape at a first environmental temperature and a second shape at a second environmental temperature, the second environmental temperature being greater than the first environmental temperature.

In any of the embodiments disclosed herein, the mold can comprise one or more patterns having features of at least approximately 5 microns or greater.

In any of the embodiments disclosed herein, the mold can comprise a surface having a treatment thereon, the treatment comprising a nonfouling release agent.

In any of the embodiments disclosed herein, the cutting can comprise laser cutting the shape memory elastomer into a form factor.

In any of the embodiments disclosed herein, the form factor can be a sheet, a membrane, a mesh, a sponge, a patch, a molded medical device, or a combination thereof.

In any of the embodiments disclosed herein, the shape memory elastomer can have a melt transition temperature from approximately 25° C. to approximately 45° C., and the melt transition temperature is greater or equal to the first environmental temperature and less than the second environmental temperature.

In any of the embodiments disclosed herein, the melt transition temperature can be from approximately 31° C. to 35° C.

In any of the embodiments disclosed herein, the shape memory elastomer can be an elastomer in the first shape and a thermoplastic in the second shape.

In any of the embodiments disclosed herein, the first shape can be a curved shape.

In any of the embodiments disclosed herein, the second shape can be a tubular shape comprising a cut along a longitudinal axis of the implant material.

In any of the embodiments disclosed herein, the shape memory elastomer can further comprise a functionalized surface.

In any of the embodiments disclosed herein, the functionalized surface can comprise a plurality of suture holes laser cut into the functionalized surface.

In any of the embodiments disclosed herein, the functionalized surface can comprise at least one functional group bonded to the shape memory polymer.

In any of the embodiments disclosed herein, the at least one functional group can comprise a bioactive agent.

In any of the embodiments disclosed herein, the molar ratio of the at least one monomer unit of glycerol to the at least one monomer unit of dodecanedioate can be from approximately 10:1 to approximately 1:10.

In any of the embodiments disclosed herein, the implant material can have a biodegradation time when implanted in vivo from approximately 4 months to approximately 24 months.

Another embodiment of the present disclosure can provide the implant material made according to the method of any of the embodiments disclosed herein.

These and other aspects of the present disclosure are described in the Detailed Description below and the accompanying figures. Other aspects and features of embodiments of the present disclosure will become apparent to those of ordinary skill in the art upon reviewing the following description of specific, exemplary embodiments of the present invention in concert with the figures. While features of the present disclosure may be discussed relative to certain embodiments and figures, all embodiments of the present disclosure can include one or more of the features discussed herein. Further, while one or more embodiments may be discussed as having certain advantageous features, one or more of such features may also be used with the various embodiments of the invention discussed herein. In similar fashion, while exemplary embodiments may be discussed below as device, system, or method embodiments, it is to be understood that such exemplary embodiments can be implemented in various devices, systems, and methods of the present disclosure.

### BRIEF DESCRIPTION OF THE DRAWINGS

The accompanying drawings, which are incorporated in and constitute a part of this specification, illustrate multiple embodiments of the presently disclosed subject matter and serve to explain the principles of the presently disclosed subject matter. The drawings are not intended to limit the scope of the presently disclosed subject matter in any manner.

FIG. 1 illustrates an implant material having a first shape and a second shape in accordance with the present disclosure.

FIG. 2 illustrates a flowchart of a method of making an implant material in accordance with the present disclosure.

FIG. 3 illustrates a flowchart of another method of making an implant material in accordance with the present disclosure.

FIG. 4 illustrates a reaction scheme for a method of making an implant material in accordance with the present disclosure.

FIGS. 5A and 5B illustrate melt transition curves for examples of an implant material in accordance with the present disclosure.

FIGS. 6A and 6B illustrate examples of an implant material having a first shape and a second shape in accordance with the present disclosure.

FIGS. 7A to 7C illustrate diagrams of the stress on an implant material in accordance with the present disclosure.

FIG. 8 illustrates a scanning electron microscope (SEM) image of an implant material in accordance with the present disclosure.

FIG. 9 illustrates a Fourier-transform infrared (FTIR) spectrum of an implant material in accordance with the present disclosure.

FIG. 10 illustrates melt transition curves for examples of an implant material in accordance with the present disclosure.

FIGS. 11A and 11B illustrate peak melt temperature and enthalpy of fusion, respectively, for implant materials in accordance with the present disclosure.

FIG. 12 illustrates photographs of examples of an implant material in accordance with the present disclosure.

FIGS. 13A and 13B illustrate volume loss and mass loss, respectively, for an implant material when implanted in vivo in accordance with the present disclosure.

FIG. 14 illustrates SEM images of an implant material after being implanted in vivo in accordance with the present disclosure.

FIG. 15 illustrates an FTIR spectra of an explanted implant material in accordance with the present disclosure.

### DETAILED DESCRIPTION

Disclosed herein are biodegradable polymers having the ability to (i) heal structural pathologies through minimally invasive interventions and (ii) support the tissue healing process requires the use thereof. Such biodegradable polymers can be suited to accommodate the mechanical requirements of the structural defect, demonstrate the ability to fully endothelialize, degrade into biocompatible byproducts, accommodate minimally invasive delivery, and support tissue and cell activity driving the replacement or repair of pathological tissues.

Suitable examples of synthetic bioresorbable polymers for numerous cardiovascular applications can generally be of the α—hydroxy ester family of biodegradable polymers. Such polymers can include, but are not limited to, poly(L-Lactide), poly(D-Lactide), poly(lactide-co-glycolide), polycaprolactone, polyurethane, and polydioxanone. Numerous challenges with minimally invasive cardiovascular repair warrant the development of resorbable materials, such as those described herein, to meet various cardiovascular tissue engineering challenges.

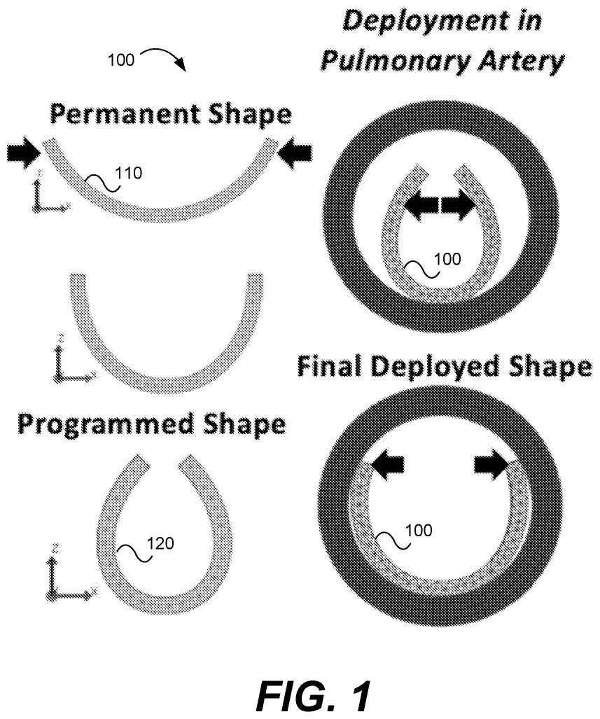

Shape memory polymers are a promising class of biomaterials with unique advantages in minimally invasive procedures. Shape memory materials are manufactured to have a permanent shape; programmed under a specific physical (light, heat, electrical) or chemical (pH) stimulus; undergo a shape change under mechanical deformation to fit within a catheter sheath; maintain this deformed shape change when the stimulus is removed; and regain the original permanent shape when the stimulus is reapplied (FIG. 1). For thermally activated shape memory polymers triggered by temperatures around 37° C., for instance, the shape memory cycle can allow deformation and programming of larger profile devices in the transitioned state to be loaded into low profile delivery devices. Upon delivery thereof into the target space, the shape memory polymers can expand to the original permanent shape. For biomedical applications, the triggering temperature can be designed to be near body temperature (37° C.) but well below the tissue necrosis temperature of 50° C. Shape memory polymers can comprise polyurethane foams, which are also approved for aneurysm occlusion with various biodegradable formulations in the pipeline.

Biodegradable shape memory polymers are a subclass of shape memory materials exhibiting shape memory properties capable of biodegradation over a prescribed timeframe. Within this subclass of polymers, biodegradable shape memory elastomers able to withstand high strains under loading can be used for cardiovascular tissue engineering. A number of these shape memory polymers can be derived from α—hydroxy ester chemistries. However, such polymers must still retain suitable mechanical properties, degradation properties, inflammatory response, and the ability to fully endothelialize and support cell activity. Additionally, such polymers utilize complex synthetic schemes with organic solvents which degrade into byproducts that elevate pH, thereby contributing to acute and chronic inflammation.

The disclosed biodegradable shape memory polymers can be synthesized from bioavailable glycerol and dodecanedioic acid. The resulting shape memory polymers can exhibit rigid elastic-plastic behavior at room temperature and have elastomeric properties above the transition temperature. The polycondensation reaction and solvent free synthesis can be attributes promoting biocompatibility. Tunability of such a polymer to achieve nonlinear mechanical characteristics of various cardiovascular tissues can be achieved via modification of the crosslink density and incorporation of composites within the polymer network. Additionally, degradation properties investigated in a mouse subcutaneous model revealed 25-50 % mass loss over the course of 4 months. The pulmonary artery can be selected as a target site for straightforward access via catheter and reduced risk for device embolization.

The shape memory polymers as described herein can be designed as a patch for percutaneous delivery and successful deployment in a pulmonary artery. The present disclosure can also assess the ability of the patch to fully endothelialize and degrade, investigate cell and tissue ingrowth, assess biocompatibility, and evaluate intrinsic material properties post-degradation.

As with various thermoset elastomeric polymers and rubbers, manufacturing PGD into complex 3D shapes is non-trivial. Key challenges with injection molding thin-walled elastomers can include resin drift and release from molds. Also disclosed herein are methods to process PGD into a thin-walled patch (approximately 0.4 mm to approximately 1 mm) with a degree of curvature to meet the design requirements for percutaneous delivery and the appropriate geometries for deployment. Also disclosed herein is a process to pattern the PGD surface without significantly impacting intrinsic material properties. Patterning of cardiovascular tissues and alignment of tissues are important attributes to aid functional tissue regeneration. Also disclosed herein is a manufacturing process to form tubular structures for delivery within the endovascular space. Complete endothelialization of implants is an important design requirement for both vascular and structural repair applications. The formation of a neointimal layer on the lumenal surface of the material implant can secure the implant in position, and also prevent embolization of exposed eroding surfaces. Correctly sized PGD patches having surface patterns can exhibit good apposition to the vascular wall, fully endothelialize, and support tissue ingrowth during degradation.

Although certain embodiments of the disclosure are explained in detail, it is to be understood that other embodiments are contemplated. Accordingly, it is not intended that the disclosure is limited in its scope to the details of construction and arrangement of components set forth in the following description or illustrated in the drawings. Other embodiments of the disclosure are capable of being practiced or carried out in various ways. Also, in describing the embodiments, specific terminology will be resorted to for the sake of clarity. It is intended that each term contemplates its broadest meaning as understood by those skilled in the art and includes all technical equivalents which operate in a similar manner to accomplish a similar purpose.

Herein, the use of terms such as “having,” “has,” “including,” or “includes” are open-ended and are intended to have the same meaning as terms such as “comprising” or “comprises”and not preclude the presence of other structure, material, or acts. Similarly, though the use of terms such as “can” or “may” are intended to be open-ended and to reflect that structure, material, or acts are not necessary, the failure to use such terms is not intended to reflect that structure, material, or acts are essential. To the extent that structure, material, or acts are presently considered to be essential, they are identified as such.

By “comprising” or “containing” or “including” is meant that at least the named compound, element, particle, or method step is present in the composition or article or method, but does not exclude the presence of other compounds, materials, particles, method steps, even if the other such compounds, material, particles, method steps have the same function as what is named.

It is also to be understood that the mention of one or more method steps does not preclude the presence of additional method steps or intervening method steps between those steps expressly identified.

The components described hereinafter as making up various elements of the disclosure are intended to be illustrative and not restrictive. Many suitable components that would perform the same or similar functions as the components described herein are intended to be embraced within the scope of the disclosure. Such other components not described herein can include, but are not limited to, for example, similar components that are developed after development of the presently disclosed subject matter.

The term “aliphatic” or “aliphatic group,” as used herein, means a straight-chain (i.e., unbranched) or branched, substituted or unsubstituted hydrocarbon chain that is completely saturated or that contains one or more units of unsaturation, or a monocyclic hydrocarbon, bicyclic hydrocarbon, or tricyclic hydrocarbon that is completely saturated or that contains one or more units of unsaturation, but which is not aromatic (also referred to herein as “carbocycle,” “cycloaliphatic” or “cycloalkyl”), that has a single point of attachment to the rest of the molecule. Unless otherwise specified, aliphatic groups contain 1-30 aliphatic carbon atoms. In some embodiments, aliphatic groups contain 1-20 aliphatic carbon atoms. In other embodiments, aliphatic groups contain 1-10 aliphatic carbon atoms. In still other embodiments, aliphatic groups contain 1-6 aliphatic carbon atoms, and in yet other embodiments, aliphatic groups contain 1, 2, 3, or 4 aliphatic carbon atoms. Suitable aliphatic groups include, but are not limited to, linear or branched, substituted or unsubstituted alkyl, alkenyl, alkynyl groups and hybrids thereof such as (cycloalkyl)alkyl, (cycloalkenyl)alkyl or (cycloalkyl)alkenyl.

The term “cycloaliphatic,” as used herein, refers to saturated or partially unsaturated cyclic aliphatic monocyclic, bicyclic, or polycyclic ring systems, as described herein, having from 3 to 14 members, wherein the aliphatic ring system is optionally substituted as defined above and described herein. Cycloaliphatic groups include, without limitation, cyclopropyl, cyclobutyl, cyclopentyl, cyclopentenyl, cyclohexyl, cyclohexenyl, cycloheptyl, cycloheptenyl, cyclooctyl, cyclooctenyl, norbornyl, adamantyl, and cyclooctadienyl. In some embodiments, the cycloalkyl has 3-6 carbons. The terms “cycloaliphatic,” may also include aliphatic rings that are fused to one or more aromatic or nonaromatic rings, such as decahydronaphthyl or tetrahydronaphthyl, where the radical or point of attachment is on the aliphatic ring. In some embodiments, a carbocyclic group is bicyclic. In some embodiments, a ‘carbocyclic group is tricyclic. In some embodiments, a carbocyclic group is polycyclic. In some embodiments, “cycloaliphatic” (or “carbocycle” or “cycloalkyl”) refers to a monocyclic C3-C6 hydrocarbon, or a C8-C10 bicyclic hydrocarbon that is completely saturated or that contains one or more units of unsaturation, but which is not aromatic, that has a single point of attachment to the rest of the molecule, or a C9-C16 tricyclic hydrocarbon that is completely saturated or that contains one or more units of unsaturation, but which is not aromatic, that has a single point of attachment to the rest of the molecule.

As used herein, the term “alkyl” is given its ordinary meaning in the art and may include saturated aliphatic groups, including straight-chain alkyl groups, branched-chain alkyl groups, cycloalkyl (alicyclic) groups, alkyl substituted cycloalkyl groups, and cycloalkyl substituted alkyl groups. In certain embodiments, a straight chain or branched chain alkyl has 1-20 carbon atoms in its backbone (e.g., C1-C20 for straight chain, C2-C20 for branched chain), and alternatively, 1-10 carbon atoms, or 1 to 6 carbon atoms. In some embodiments, a cycloalkyl ring has from 3-10 carbon atoms in their ring structure where such rings are monocyclic or bicyclic, and alternatively 5, 6 or 7 carbons in the ring structure. In some embodiments, an alkyl group may be a lower alkyl group, wherein a lower alkyl group comprises 1-4 carbon atoms (e.g., C1-C4 for straight chain lower alkyls).

As used herein, the term “alkenyl” refers to an alkyl group, as defined herein, having one or more double bonds.

As used herein, the term “alkynyl” refers to an alkyl group, as defined herein, having one or more triple bonds.

As used herein, the term “azide” is given its ordinary meaning in the art and may include an alkyl group, as defined herein, having one or more azide functional groups.

The term “heteroalkyl” is given its ordinary meaning in the art and refers to alkyl groups as described herein in which one or more carbon atoms is replaced with a heteroatom (e.g., oxygen, nitrogen, sulfur, and the like). Examples of heteroalkyl groups include, but are not limited to, alkoxy, poly(ethylene glycol), alkyl-substituted amino, tetrahydrofuranyl, piperidinyl, morpholinyl, etc.

The term “aryl” used alone or as part of a larger moiety as in “aralkyl,” “aralkoxy,” or “aryloxyalkyl,” refers to monocyclic or bicyclic ring systems having a total of five to fourteen ring members, wherein at least one ring in the system is aromatic and wherein each ring in the system contains 3 to 7 ring members. The term “aryl” may be used interchangeably with the term “aryl ring.” In certain embodiments of the present invention, “aryl” refers to an aromatic ring system which includes, but not limited to, phenyl, biphenyl, naphthyl, binaphthyl, anthracyi and the like, which may bear one or more substituents. Also included within the scope of the term “aryl,” as it is used herein, is a group in which an aromatic ring is fused to one or more non-aromatic rings, such as indanyl, phthalimidyl, naphthimidyl, phenanthridinyl, or tetrahydronaphthyl, and the like.

The terms “heteroaryl” and “heteroar-,” used alone of as part of a larger moiety, e.g., “heteroaralkyl,” or “heteroaralkoxy,” refer to groups having 5 to 10 ring atoms (i.e., monocyclic or bicyclic), in some embodiments 5, 6, 9, or 10 ring atoms. In some embodiments, such rings have 6, 10, or 14 π electrons shared in a cyclic array; and having, in addition to carbon atoms, from one to five heteroatoms. The term “heteroatom” refers to nitrogen, oxygen, or sulfur, and includes any oxidized form of nitrogen or sulfur, and any quatemized form of a basic nitrogen. Heteroaryl groups include, without limitation, thienyl, furanyl, pyrrolyl, imidazolyl, pyrazolyl, triazolyl, tetrazolyl, oxazolyl, isoxazolyl, oxadiazolyl, thiazolyl, isothiazolyl, thiadiazolyl, pyridyl, pyridazinyl, pyrimidinyl, pyrazinyl, indolizinyl, purinyl, naphthyridinyl, and pteridinyl. In some embodiments, a heteroaryl is a heterobiaryl group, such as bipyridyl and the like. The terms “heteroaryl” and “heteroar-,” as used herein, also include groups in which a heteroaromatic ring is fused to one or more aryl, cycloaliphatic, or heterocyclyl rings, where the radical or point of attachment is on the heteroaromatic ring. Nonlimiting examples include indolyl, isoindolyl, benzothienyl, benzofuranyl, dibenzofuranyl, indazolyl, benzimidazolyl, benzthiazolyl, quinolyl, isoquinolyl, cinnolinyl, phthalazinyl, quinazolinyl, quinoxalinyl, 4H—quinolizinyl, carbazolyl, acridinyl, phenazinyl, phenothiazinyl, phenoxazinyl, tetrahydroquinolinyl, tetrahydroisoquinolinyl, and pyrido[2,3-b]-1,4-oxazin-3(4H)-one. A heteroaryl group may be monocyclic, bicyclic, tricyclic, tetracyclic, and/or otherwise polycyclic. The term “heteroaryl” may be used interchangeably with the terms “heteroaryl ring,” “heteroaryl group,” or “heteroaromatic,” any of which terms include rings that are optionally substituted. The term “heteroaralkyl” refers to an alkyl group substituted by a heteroaryl, wherein the alkyl and heteroaryl portions independently are optionally substituted.

As used herein, the terms “heterocycle,” “heterocyclyl,” “heterocyclic radical,” and “heterocyclic ring” are used interchangeably and refer to a stable 5- to 7-membered monocyclic or 7-10-membered bicyclic heterocyclic moiety that is either saturated or partially unsaturated, and having, in addition to carbon atoms, one or more, preferably one to four, heteroatoms, as defined above. When used in reference to a ring atom of a heterocycle, the term “nitrogen” includes a substituted nitrogen.

A heterocyclic ring can be attached to its pendant group at any heteroatom or carbon atom that results in a stable structure and any of the ring atoms can be optionally substituted. Examples of such saturated or partially unsaturated heterocyclic radicals include, without limitation, tetrahydrofuranyl, tetrahydrothiophenyl pyrrolidinyl, piperidinyl, pyrrolinyl, tetrahydroquinolinyl, tetrahydroisoquinolinyl, decahydroquinolinyl, oxazolidinyl, piperazinyl, dioxanyl, dioxolanyl, diazepinyl, oxazepinyl, thiazepinyl, morpholinyl, and quinuclidinyl. The terms “heterocycle,” “heterocyclyl,” “heterocyclyl ring,” “heterocyclic group,” “heterocyclic moiety,” and “heterocyclic radical,” are used interchangeably herein, and also include groups in which a heterocyclyl ring is fused to one or more aryl, heteroaryl, or cycloaliphatic rings, such as indolinyl, 3H-indolyl, chromanyl, phenanthridinyl, or tetrahydroquinolinyl. A heterocyclyl group may be monocyclic, bicyclic, tricyclic, tetracyclic, and/or otherwise polycyclic. The term “heterocyclylalkyl” refers to an alkyl group substituted by a heterocyclyl, wherein the alkyl and heterocyclyl portions independently are optionally substituted.

As used herein, the term “partially unsaturated” refers to a ring moiety that includes at least one double or triple bond. The term “partially unsaturated” is intended to encompass rings having multiple sites of unsaturation but is not intended to include aryl or heteroaryl moieties, as herein defined.

The term “heteroatom” means one or more of oxygen, sulfur, nitrogen, phosphorus, or silicon (including, any oxidized form of nitrogen, sulfur, phosphorus, or silicon; the quaternized form of any basic nitrogen or; a substitutable nitrogen of a heterocyclic ring.

The term “unsaturated,” as used herein, means that a moiety has one or more units of unsaturation.

The term “halogen” means F, Cl, Br, or I; the term “halide” refers to a halogen radical or substituent, namely —F, —Cl, —Br, or —I.

As described herein, compounds of the invention may contain “optionally substituted” moieties. In general, the term “substituted,” whether preceded by the term “optionally” or not, means that one or more hydrogens of the designated moiety are replaced with a suitable substituent. Unless otherwise indicated, an “optionally substituted” group may have a suitable substituent at each substitutable position of the group, and when more than one position in any given structure may be substituted with more than one substituent selected from a specified group, the substituent may be either the same or different at every position. Combinations of substituents envisioned by this invention are preferably those that result in the formation of stable or chemically feasible compounds. The term “stable,” as used herein, refers to compounds that are not substantially altered when subjected to conditions to allow for their production, detection, and, in certain embodiments, their recovery, purification, and use for one or more of the purposes disclosed herein.

The term “spiro compound” refers to a chemical compound that presents a twisted structure of two or more rings, in which at least 2 rings are linked together by one common atom, e.g., a carbon atom. When the common atom is located in the center of the compound, the compound is referred to as a “spirocentric compound.” The common atom that connects the two or more rings is referred to as the “spiro-atom.” When such common atom is a carbon atom, it is referred to as the “spiro-carbon.”

Unless otherwise stated, all tautomeric forms of the compounds of the invention are within the scope of the invention.

Additionally, unless otherwise stated, structures depicted herein are also meant to include compounds that differ only in the presence of one or more isotopically enriched atoms. For example, compounds having the present structures except for the replacement of hydrogen by deuterium or tritium, or the replacement of a carbon by a 11C- or 13C- or 14C-enriched carbon are within the scope of this invention.

Reference will now be made in detail to exemplary embodiments of the disclosed technology, examples of which are illustrated in the accompanying drawings and disclosed herein. Wherever convenient, the same references numbers will be used throughout the drawings to refer to the same or like parts.

FIG. 1 illustrates an implant material 100 having a first shape 110 and a second shape 120. The implant material 100 can comprise a shape memory polymer, and the shape memory polymer can confer properties of the first shape 110 and the second shape 120 to the implant material 100. The shape memory polymer can comprise at least one monomer unit of glycerol and at least one monomer unit of dodecanedioate. Additional other monomer units can be present in the shape memory polymer as desired. Additives can also be added to the shape memory polymer, such as porogens, surfactants, binders, emulsifiers, and the like.

The ratio of the glycerol to the dodecandeioate can be altered as desired to confer certain properties to the implant material 100. For example, the molar ratio of the at least one monomer unit of glycerol to the at least one monomer unit of dodecanedioate can be from approximately 10:1 to approximately 1:10 (e.g., from 9:1 to 1:10, from 8:1 to 1:10, from 7:1 to 1:10, from 6:1 to 1:10, from 5:1 to 1:10, from 4:1 to 1:10, from 3:1 to 1:10, from 2:1 to 1:10, from 1:1 to 1:10, from 10:1 to 1:9, from 10:1 to 1:8, from 10:1 to 1:7, from 10:1 to 1:6, from 10:1 to 1:5, from 10:1 to 1:4, from 10:1 to 1:3, from 10:1 to 1:2, from 10:1 to 1:1, or from 5:1 to 1:5).

The shape memory polymer can also have a functionalized surface. The functionalized surface can comprise a plurality of suture holes laser cut into the functionalized surface. The suture holes, or other surface modulations, can be implemented in the implant device 100 to alter the mechanical properties of the implant device 100 as desired. The functionalized surface can comprise, for example, suture holes, grooves, wells, ribs, raised portions, other patterns, and the like. The patterns in the functionalized surface can be patterned using subtractive manufacturing. The functionalized surface can also comprise at least one functional group bonded to the shape memory polymer. The functionalized surface can be conjugated to improve the biocompatability of the implant material 100. For example, the at least one functional group can be a bioactive agent. The functionalized surface can also be altered to include a plurality of living and/or nonliving cells.

The shape memory polymer can take the first shape 110 at a first environmental temperature and the second shape 120 at a second environmental temperature. The shape memory polymer can have a melt transition temperature from approximately 25° C. to approximately 45° C.(e.g., from 26° C. to 44° C., from 27° C. to 43° C., from 28° C. to 42° C., from 29° C. to 41° C., from 30° C. to 40° C., from 31° C. to 39° C., from 32° C. to 38° C., from 33° C. to 37° C. from 34° C. to 36° C., from 30° C. to 39° C., from 31° C. to 38° C., from 31° C. to 37° C., from 31° C. to 36° C., or from 31° C. to 35° C.). The melt transition temperature can be greater than or equal to the first environmental temperature and less than the second environmental temperature. Alternatively, or in addition, the shape memory polymer can be an elastomer above the melt transition temperature and a thermoplastic in the below the melt transition temperature.

The first shape 110 can be considered a “permanent” or recovered shape. In other words, the implant material 100 can be configured to return to the first shape when no stimulus is present. The first shape 110 can be a curved or tubular shape. The second shape 120 can be a “programmed” or stimulated shape. In other words, the implant material 100 can be configured to take the second shape 120 in response to a stimulus, such as temperature, light, pH, and the like. For example, the implant material 100 can take the second shape in response to a temperature stimulus of the second environmental temperature being greater than the first environmental temperature. The second shape 120 can be a tubular shape comprising a cut along a longitudinal axis of the implant material 100.

By way of another example, the permanent shape can be a curve and the programmed shape can be tubular. When subjected to stimulus of a cold temperature, the implant material 100 can take the programmed shape and behave as a thermoplastic below the melt transition temperature. When implanted into a warm environment, the implant material 100 can recover to the permanent shape when implanted and behave as an elastomer above the melt transition temperature.

Furthermore, the implant material can have a biodegradation time when implanted in vivo from approximately 4 months to approximately 24 months (e.g., from 5 months to 23 months, from 6 months to 22 months, from 7 months to 21 months, from 8 months to 21 months, from 9 months to 20 months, from 10 months to 20 months, from 10 months to 15 months, from 5 months to 15 months, from 4 months to 10 months, or from 12 months to 24 months).

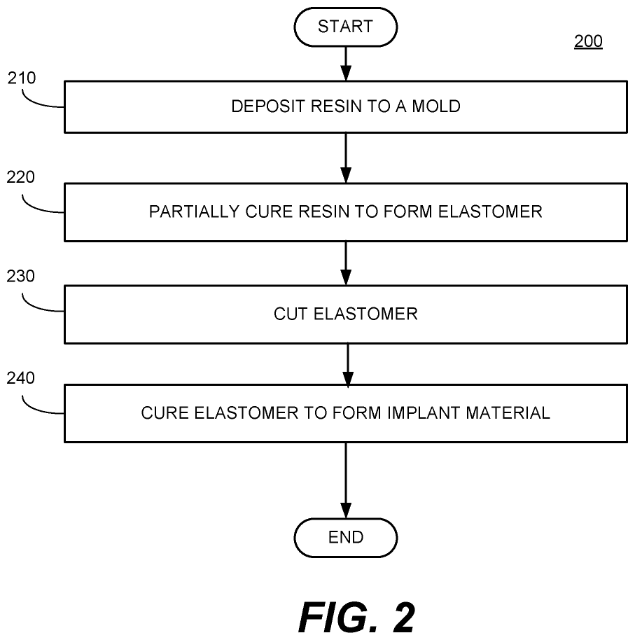

FIG. 2 is a flowchart of a method 200 of making an implant material 100. As shown in block 210, the shape memory polymer can be deposited in resin form into a mold. The mold can be 3D printed, and the mold can have various patterns to impart surface geometry to the functionalized surface of the implant material 100. The various patterns in the mold can have features having a size of approximately 5 microns or greater. The mold can also be surface treated with a nonfouling release agent, such as parylene. The method 200 can then proceed on to block 220.

In block 220, the shape memory polymer can be partially cured to move from the resin form to an elastomer form in the mold. The shape memory elastomer can also be released from the mold. As would be appreciated, the surface treatment of the mold can aid in releasing the shape memory elastomer from the mold. The method 200 can then proceed on to block 230.

In block 230, the partially cured shape memory elastomer can be cut. For example, the shape memory elastomer can be laser cut into a variety of shapes as desired, such as a patterned mesh. The cut shape memory elastomer can further have its surface geometry altered by subtractive manufacturing to impart additional patterns to the shape memory polymer. The various patterns added by subtractive manufacturing can have features having a size of approximately 5 microns or greater. The method 200 can then proceed on to block 240.

In block 240, the shape memory elastomer can be fully cured to form the implant material 100. The implant material 100 can be fully cured at a variety of temperatures, temporal durations, and pressures as desired to yield desirable properties in the implant material 100, such as crosslink density and toughness. The shape memory elastomer can also be fully cured in a permanent shape such that the implant material 100 takes on a permanent shape. For example, the shape memory elastomer can be fully cured on a roller such that the implant material 100 is a tubular shape. The method 200 can terminate after block 240. However, the method 200 can also proceed on to other method steps not shown.

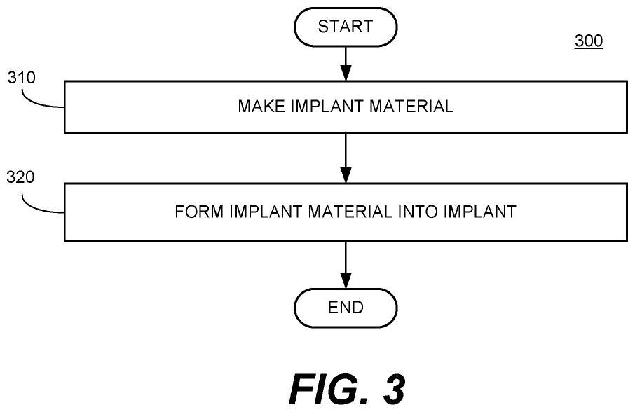

FIG. 3 is a flowchart of another method 300 of making an implant material 100. In block 310, the implant material 100 can be made through any of the methodologies as described herein. In block 320, the implant material 100 can be formed into an implant. The implant can be in the form of a sheet, a membrane, a mesh, a sponge, a patch, a molded medical device, or combinations thereof. The implant can also be functionalized as described herein such that the implant has a functionalized surface.

Certain embodiments and implementations of the disclosed technology are described above with reference to block and flow diagrams of systems and methods and/or computer program products according to example embodiments or implementations of the disclosed technology. It will be understood that one or more blocks of the block diagrams and flow diagrams, and combinations of blocks in the block diagrams and flow diagrams, respectively, can be implemented by computer-executable program instructions. Likewise, some blocks of the block diagrams and flow diagrams may not necessarily need to be performed in the order presented, may be repeated, or may not necessarily need to be performed at all, according to some embodiments or implementations of the disclosed technology.

### EXAMPLES

The following examples are provided by way of illustration but not by way of limitation.

Poly(glycerol dodecanedioate) (PGD) can exhibit tunable transition temperatures around body temperature (34-38° C.), nonlinear elastic properties approximating cardiac tissues, and favorable degradation rates in vitro. Degree of endothelialization; degradation and consequent changes in polymer thermomechanical properties; and inflammatory response in preclinical animal models are unknown material attributes for translating such a material into cardiovascular devices. The present disclosure can assess changes in polymer structure, endothelialization, inflammation and tissue ingrowth of percutaneously implanted PGD patches(20 mm×9 mm×0.5 mm) into the branch pulmonary arteries of Yorkshire pigs for 3 months. After 3 months in vivo, all samples can be endothelialized, with ⅝ samples exhibiting (100%) endothelialization, 2/8 samples exhibiting 85-95% endothelialization, and ⅛ samples exhibiting limited (<20%) endothelial coverage with mild-moderate inflammation. PGD explants exhibited a (60-70%) volume loss and (25-30%) mass loss and a reduction in polymer crosslinks. Lumenal and mural surfaces, and the cross section of the explant demonstrated evidence of degradation, tissue ingrowth and cellular growth. Therefore, PGD is an appropriate cardiovascular engineering material due to its propensity for rapid endothelialization and uneventful inflammatory response in a preclinical animal model; establishing a use in cardiovascular repair applications.

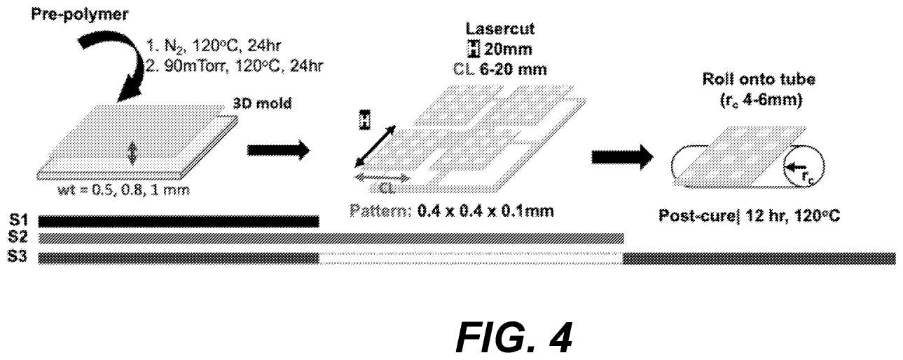

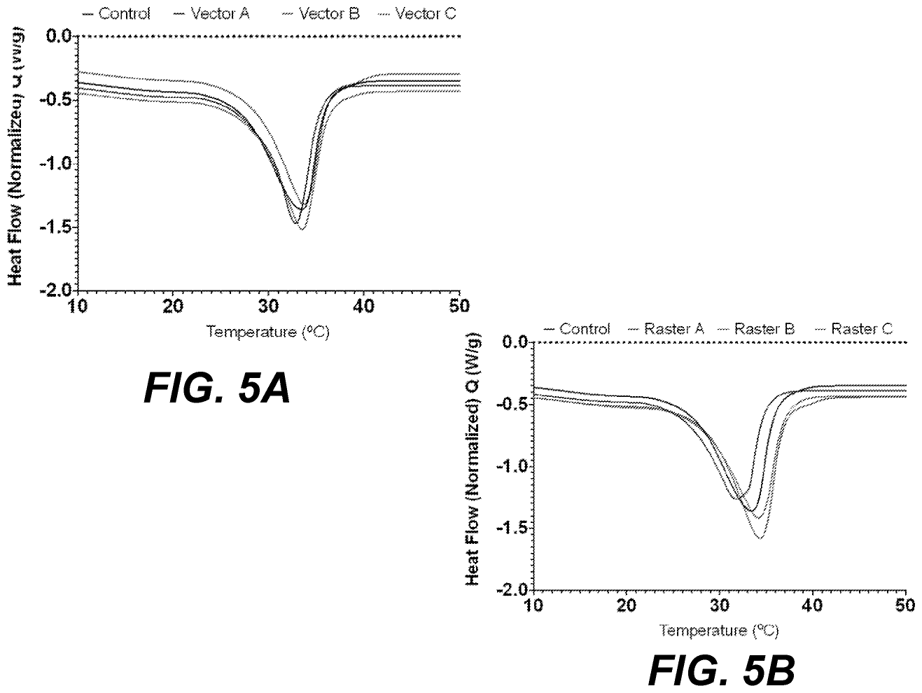

PGD prepolymer can be synthesized as previously described. Briefly, equal amounts of dodecanedioic acid (Sigma-Aldrich, St. Louis MO) and glycerol (MP Biomedical, LLC, Solon OH) can be mixed at 120° C. under nitrogen for 24 h and for another 24 h at 120° C. under vacuum at 90 mTorr. Manufacturing processes as described herein can be used to create tubular, thin walled, patterned constructs (FIG. 4). Polymer can be poured in autoclaved rectangular 3D printed (Form2 FormLabs) molds of varying thickness (0.5, 0.8, 1 mm) which can be cured for varying times (S1:24 h, S2-36 h, S3-24 h) under 90 mTorr vacuum at 120° C. Partially cured (S₁) and single step (S₂) cured samples can be removed from the mold and stored in a vacuum desiccator until further evaluation. Two-step cured samples (S₃) can be lasercut to size (20 mm H×6-20 mm CL) using a CO₂ laser cutter and the vector cut feature making 2 cuts at 80% intensity. Two-step cured samples (S3) can be patterned using a raster cut feature at 20-40% intensity with 4 passes at 5 focal depths. Changes in intrinsic polymer properties were measured after laser cutting (FIGS. 5A and 5B). Samples can then be wrapped around a 3D printed tube having the desired radius of curvature (4-6 mm), and fully cured for an additional 12 h at 120° C. to create constructs having a permanent curved shape.

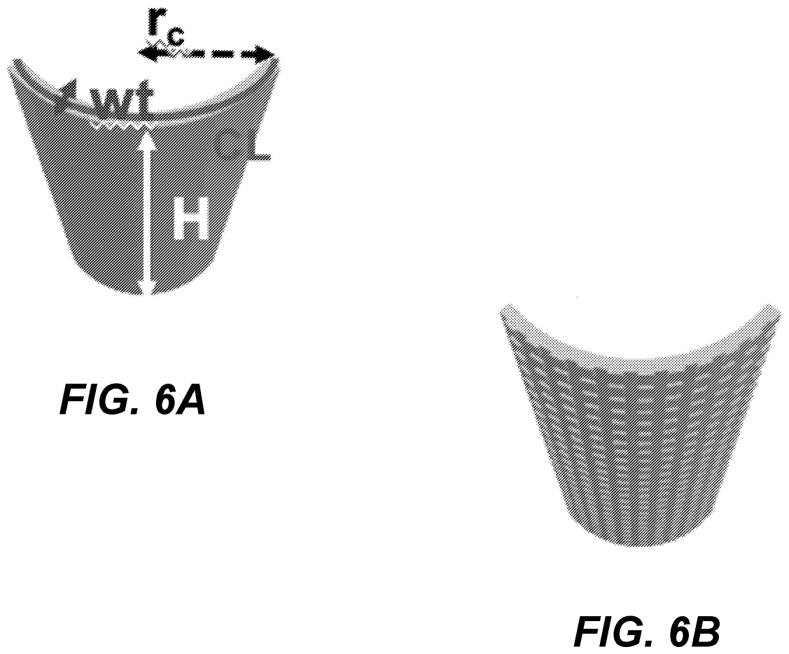

Pulmonary artery patch design: Porcine pulmonary artery dimensions can be approximated from the geometric shape of porcine pulmonary arteries of pigs at different body weights. Placement of the devices can be targeted for the proximal branch of the main artery or moderately distal in the smaller right (inner diameter 6.2-11 mm) or left (inner diameter 5.8-9 mm) arterial branches. Pulmonary patch specifications can also be constrained by permissible catheter geometries. The cross-sectional area of the delivery sheaths can be used as a constraint for designing the pulmonary artery patch material. Resulting patch designs can have a height of 20 mm, CL of 6-20 mm, wt of 0.5-1 mm, and a radius of curvature (ROC) of 4-6 mm (FIG. 6A). Finally, 400×400×100 μm posts can be included in the designs to determine if the patterned materials could improve surface roughness and increase forces able to resist dislodgement (FIG. 6B).

Finite element modeling of PGD arterial patch deployment: The Fung-Choung and Holzapfel-Gasser-Ogden along with orthotopic solid mixture variants of these models can be constitutive models for pulmonary arteries. Pulmonary artery models (6-9 mm) can be created in FEBiO, and an anisotropic Holzapfel-Gasser-Odgen constitutive model can be implemented using a using an uncoupled solid mixture material with a material axis aligned along the principal cylindrical artery orientation and a Mooney Rivlin ground matrix [μ(kPa): 2.1803] with a two fiber uncoupled solid mixture [k1: (kPa) 0.0732, k2: 0.0732, φ° 35.718]. The material properties can be derived from porcine neonatal arteries to serve as a conservative lower bound for artery stiffness. PGD hyperelastic properties can be defined by a one term Ogden model [μ₁:4.29, α₁:0.111]. A deformation body force can be applied to the polymer (FIG. 1) which resulted in folding the polymer. Once the polymer is placed in the pulmonary artery model, this force can be applied in reverse to unfurl the polymer modeling recovery (FIG. 1). Stress distribution within the polymer due to programming, and stress distribution at the polymer-tissue interface after recovery can be evaluated to assess risk of implant and arterial failure.

Differential Scanning calorimetry (DSC): DSC can be conducted using a Discovery Q250 with an RCS90 cooling system (TA instruments, New Castle DE). Samples (n=4) can be dried in a vacuum desiccator, weighed, and placed in a Tzero® pan. Samples can be heated to 90° C. from 25° C. at a rate of 20° C./min to remove thermal history. After an isothermal hold for 3 min, samples can be cooled at a rate of 10° C./min from 90° C. to −50° C. After another isothermal hold for 3 min, samples can be heated from −50° C. to 70° C. at a rate of 10° C./min. Thermal transitions (peak melt transition, Tₘ and enthalpy of fusion, ΔH_{fusion} J/g) can be measured using the TRIOS version 4.4 (TA instruments) analysis software.

Optical Imaging: Images of pulmonary artery gross anatomy can be obtained using a D60 DSLR camera (Nikon, Tokyo, Japan) with a 35 mm Nikkor lens (Nikon) and a 2.3 mm F stop. Images of explants can be obtained using a D7000 DSLR camera (Nikon) with a 55 mm f/2.8 Nikkor AF Micro lens (Nikon). Optical microscopy images of explanted PGD can be captured using a Leica DVM6 (Leica Camera AG, Wetzlar, Germany). Image processing can be done using the accompanying LAS X software. Various magnifications, locations, and sample orientations can be used for each sample. PGD optical microscopy image samples can be prepared by cutting a roughly 3×6 mm specimen using a razor blade and adhering it, convex and patterned side up, to a steel SEM sample stage. Compressed air can be used to remove any surface debris. The sample can then be coated with gold using a Sputter Coater 108 (Cressington, Watford, United Kingdom) in an argon 0.08 mbar atmosphere and using a 30 mA current for 80 seconds. Optical microscopy images can be captured using a Leica DVM6 (Leica Camera AG, Wetzlar, Germany). Image processing can be done using the LAS X (Leica) analysis software.

Scanning Electron Microscopy: Samples can be prepared by cutting three roughly 2.5×1.5×1.5 mm specimens from each of the test samples using a razor blade (lumenal, mural and cross section) and placed side-by-side on a steel sample stage. SEM can be conducted on a Generation 5 Phenom ProX SEM (Thermo Fisher Scientific, Waltham MA) utilizing a backscattered electron detector (BSD) and 15 kV acceleration voltage. SEM micrographs can be captured at four locations on each of the three uniquely oriented specimens in each sample group. At each location, both high and low magnifications can be used, roughly 410× magnification for a roughly 659 μm field of view (FOV) and 1000× magnification for a 269 μm FOV. Micrographs can be captured using both the full BSD array and a partial BSD array for topographical data.

Fourier Transform Infrared Spectroscopy: PGD manufacturing analysis can be evaluated by taking three PGD specimens analyzed (each from different test group), and two small roughly 2×2×2 mm samples and spectra can be collected for the top (convex side), bottom (concave side), and cross-section. For sterilization analysis, four locations can be analyzed on each sample (1=4/surface, 1 surface/n, n=3/group for 6 total samples, 24 total spectra, 12 spectra/group i.e., 1=4/n, n=1/surface, 3 surfaces/group). For explant analysis, PGD post-implant specimens can be analyzed at four locations per sample, two from the mural side and two from the lumenal side (1=4/n, n=1/group for 4 total samples and 16 total spectra, 4/group). FTIR can be performed using a Nicolet iS5 and iD7 ATR (Thermo Fisher Scientific, Waltham MA) and data can be processed using accompanying OMNIC analysis software.

Rheometry: Rheometry can be conducted using an MCR 302 rotational rheometer (Anton Paar GmbH, Graz, Austria) equipped with a PPTD-200 and an 8 mm parallel plate sandblasted measuring set. A temperature sweep can be run on PGD samples (8×2 mm, n=3) from −10° C. to 80° C. at a constant angular frequency (w̅=10 rad/s) and a constant strain (5 %). The complex shear modulus G* and loss factor (tan δ) can be evaluated using the RheoCompass1.2 (Anton Paar) analysis software, and the transition temperatures can be calculated after the inflection point (tan δ′).

Shape recovery assessment: Shape recovery samples (30×5×2 mm, n=3) can be heated to 90° C. and bent to a 160° angle. Samples can be allowed to cool to room temperature from 90° C., and the programmed angle can be measured at a stress-free condition. Samples can then be held in a water bath at 37° C., and time to recover as well as final recovery angle can be recorded. Shape fixity (R_{f}) and shape recovery (Rᵣ) can be calculated using equations (1) and (2) but adapted to this angular recovery test.

$$\\ \begin{matrix}{R}_{f} = \frac{{θ}_{u} ( N )}{{θ}_{m}} & ( 1 )\end{matrix}$$

$$\\ \begin{matrix}{R}_{r} = \frac{{θ}_{m} - {θ}_{p} ( N )}{{θ}_{m} - {θ}_{p} ( N - 1 )} & ( 2 )\end{matrix}$$

where θₘ is the mechanical deformation causing the programming of the temporary shape θᵤ(N), θₘ−θₚ(N) is the change in angle in the course of recovery, and θₘ−θₚ(N−1) is the change in angle during the course of programming. Recovery rate (radians/min) was also calculated by

$$\\ \begin{matrix}\frac{{dR}_{r}}{dt} = \frac{{θ}_{m} - {θ}_{p} ( N )}{Δ t} . & ( 3 )\end{matrix}$$

Bench testing: PGD implants can be heated to 40° C., rolled into a tube, cooled to 4° C. and loaded into a catheter sheath. Hollow cylinders can be 3D printed using a Form 2 (Formlabs, Somerville MA) 3D printer and elastic resin to model the anticipated pulmonary artery location targets. Artery models can be immersed in a water bath at 37° C., and implants can be deployed. After deployment, models can be shaken on an agitator (300-500 RPM) to observe drift or dislodgement. Final designs can be tested in a 3D printed model of a pulmonary artery, and constant fluid flow can be applied to challenge for drift, dislodgement or occlusion.

Implantation procedure: Eight material implantations can be performed. Implant specimens can be sent to Case Western Reserve University Hospital where they can be sterilized in an ethylene oxide chamber (Steris AMSCO® Eagle® 3017 100% EO Sterilizer/Aerator, Mentor OH) at 37° C. using 100% ethylene oxide gas for 4 h, outgassed for 48 h, and stored in a vacuum desiccator until further use. Four female Yorkshire swine (20-30 kg) can be prepared and draped in sterile fashion. Percutaneous access can be obtained under ultrasound guidance initially with a 6F upsized to 12F sheath in all but one swine via the left internal jugular vein. The first case can have percutaneous access with a 6F r sheath upsized to a 14F via the right femoral vein. Following initial access, a 6F wedge catheter can be advanced as distally as possible into either the right or left lower pulmonary artery. Wedge angiography can confirm optimal catheter placement. The 6F short sheath can then be exchanged over a 0.035-inch AGA wire for a Flexor long sheath (either 14F or 12F), which can be advanced into distal position.

PGD can be warmed to approximately 40° C. and loaded into the distal end of an 11Fr short sheath, with the hub removed. The modified 11Fr sheath can be inserted into the 12F Flexor long sheath, and the 11Fr dilator (with the tip trimmed off) can be advanced through the modified 11Fr sheath to deliver the device material into the long sheath. The modified 11 Fr sheath and dilator can be removed. Next, the 12F dilator (also with the tip trimmed off) can be advanced through the long sheath, ultimately delivering the implant material into the distal pulmonary artery. Post branch pulmonary artery angiograms can be performed to confirm placement of the material for posthumous retrieval and examination. The 12F dilator can then be exchanged for the 6F wedge catheter in order to establish position in the contralateral lung, again in a lower branch, as distally as possible. Wedge angiograms confirmed optimal position. The wedge catheter can be removed, and a second material device can be prepared as noted above. It can be delivered into the pulmonary artery using the delivery system previously described. Final angiograms can be repeated before all catheters and sheaths were removed. Closing ACTs can be <200 in all cases.

Mass Loss and Swelling: Sample mass can be measured post-explanation and compared to the mass prior to implantation. Similarly, volume measurements based on circumferential length, thickness, and height of the implant can be used to estimate volume loss. Samples (18-24 mg) can be cut from explants and controls (n=3 replicates and n=2 samples/group). Samples can be immersed in 3.0 mL tetrahydrofuran (THF), weighed once more, and incubated at 37 ° C. for seven days (n=8). THF can be replenished daily for seven days, and samples can be weighed daily in between THE changes. Equilibrated swollen samples can be weighed and subsequently dried for seven days. Dried samples can be weighed. Difference in dry weight after swelling and before can be used to calculate the dissolved and uncrosslinked fraction. Difference in swollen day seven samples vs. dry samples can be used to calculate swelling percent.

Histopathology: Samples can be dissected with intact neointimal layer and mural layers. Sections of the pulmonary artery adjacent and distal to the implant can be fixed in formalin, paraffin embedded, sectioned, and stained with Hematoxylin and Eosin (n=3 slides and n=3 sections/slide). The foreign body giant cell response, inflammation score, and fibrosis scores on patterned and smooth implants can be tabulated. Areas containing lesions can be documented and annotated. Upon dissection, samples can be frozen and stored at −80° C. until further processing. Samples can be thawed and embedded in OCT so a cross sectional cut could reveal the intimal and mural layers of the artery. Sections can be mounted on slides (3 sections 20-25 μm apart per slide and 3 slides per sample). Slides can be fixed, retrieved in 0.2% Triton X, blocked in 4% goat serum, and stained using 1:200 Rhodhamine-Phalloidin (F-actin), primary 1:50 CD-31 (PECAM) rabbit polyclonal antibody (Abcam, Cambridge UK), primary 1:50 α-SMA mouse monoclonal antibody (Thermo Fisher Scientific, Waltham MA) and secondary goat anti-rabbit 1:500 Alexa 488 (Thermo Fisher Scientific) and anti-mouse 1:500 Alexa-694 (Thermo Fisher Scientific) in accordance with manufacture recommended protocols. A Zeiss 700 Laser Scanning Confocal microscope (Carl Zeiss AG, Jena, Germany) can be used to image slides using the 10×, 20×and 40× objectives. Excitation lasers at 405, 488, 555, and 663 nm can be used in conjunction with Zen 2012 software supported to collect fluorescence images. Images can be collected at a scan speed of 8 μm/s and averaged 8 times. Z-stack images can be collected over 25 μm depth with one micron interval.

Statistical methods: One-way ANOVA with a Tukey post-hoc test can be conducted for evaluating differences in Tₘ, Tₑ, ΔH_{fusion}, crosslinked fraction, swelling %, tan δ, G*, R_{f}, Rᵣ, and

$$\\ \frac{{dR}_{r}}{dt} .$$

An unpaired t-test assuming a gaussian distribution can be conducted to evaluate differences in volume and mass loss. A two-tailed Mann-Whitney test on ranks can be conducted to analyze histological scores.

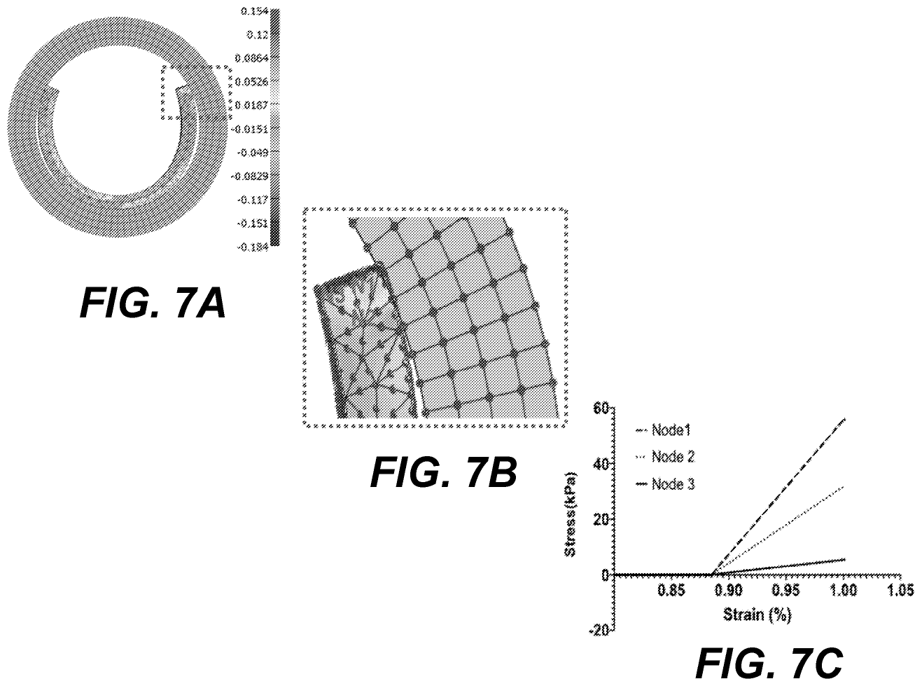

PGD pulmonary artery patches can be designed to have wider curvature than the targeted pulmonary artery to increase apposition to the arterial wall (FIG. 1). PGD patch deployment in the pulmonary artery models can avoid rupturing wall stress in nonlinear finite element simulations of patch deployment. In fact, there can be greater stress within the polymer compared to the artery wall(FIGs. 7A to 7C). There was no difference in implant stress with varying length and height, but as expected, changing the Radius of Curvature (ROC) can impact the stress when in contact with the implant. Moreover, increasing the wall thickness of the PGD implant can result in decreased implant stress. Interestingly, the patterned designs can increase stress within the implant compared to unpatterned designs. Material properties of the shape memory PGD patch, geometry of the patch, and the deployment location within the targeted pulmonary artery branch can impact successful delivery. The recovery rate (Rr) of PGD is dependent on polymer composition.

Artery tissues can exhibit non-linear viscoelasticity, which with a rapid polymer device recovery rate, can risk being damaged under a large recovery force. The actuation force of the polymer and time to recover can be determined based on recovery rate and actuation force from prior studies. Complete recovery of the patch material and strong opposition to the artery wall can be considered. Shape recovery of computational models can indicate stress within the implant during deployment does not cause device failure or arterial rupture (FIGS. 7A to 7C). As the polymer recovers due to thermal energy, it can undergo a shape and phase transition from a hard plastic to a soft elastomer. Because the radius of curvature designed in the permanent polymer shape can be greater than the target artery branch, the patch can continue to recover within the artery applying a force upon the artery wall. Intrinsic polymer properties can drive this recovery, and the force resulting from the recovery can depend on material factors including the shape memory recovery rate and initial programmed curvature.

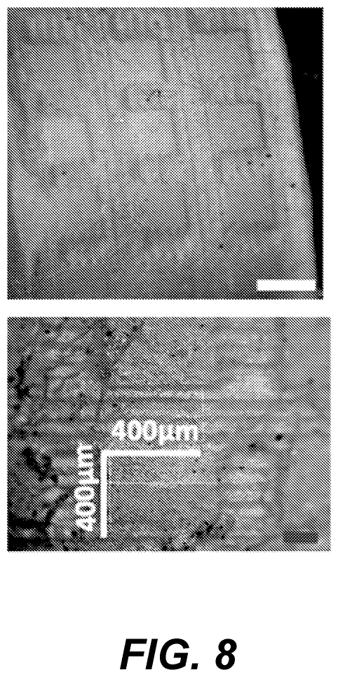

PGD prepolymer poured in Form 2 molds can spread uniformly and release upon curing (FIG. 4). The top surface of the sheet can be smooth, whereas the bottom surface can exhibit surface roughness (FIG. 4). Scanning electron micrographs of the top surfaces revealed smooth surfaces with some indents at higher magnifications where bubbles within the polymer can be drawn out during curing (FIG. 4). The bottom surface of the PGD sheet can exhibit surface roughness imparted by the roughness in the 3D printed mold. The in-plane resolution of the Form2 SLA printer can be 50 microns and can be largely governed by the point spread function of the UV laser intensity. However, there can be a tradeoff between roughness and release. Creating posts or indents having larger dimensions in the molds can result in poor release. Therefore, a combination of casting and laser cutting can be explored to create surface characteristics at varying length scales. SEM micrographs of laser cut PGD can reveal adherence to overall design geometry and raster cutting patterns revealed surface micropatterns having intended design attributes (FIG. 8).

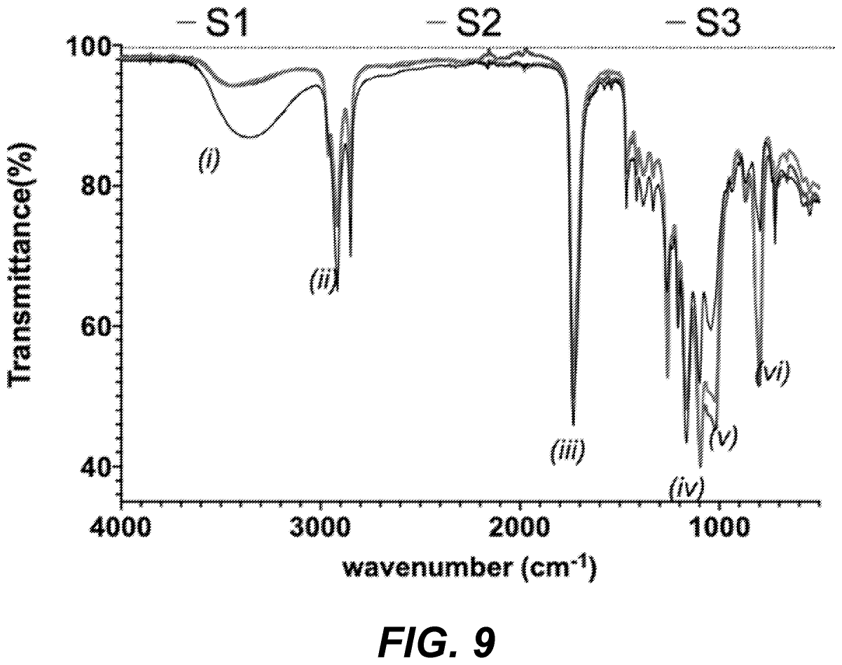

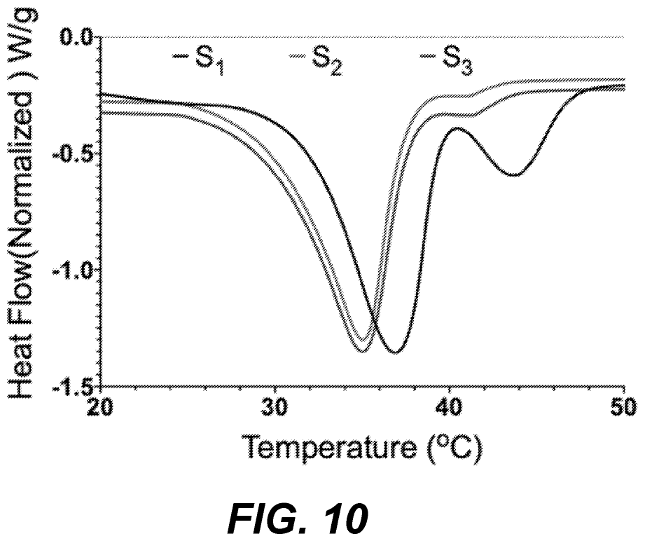

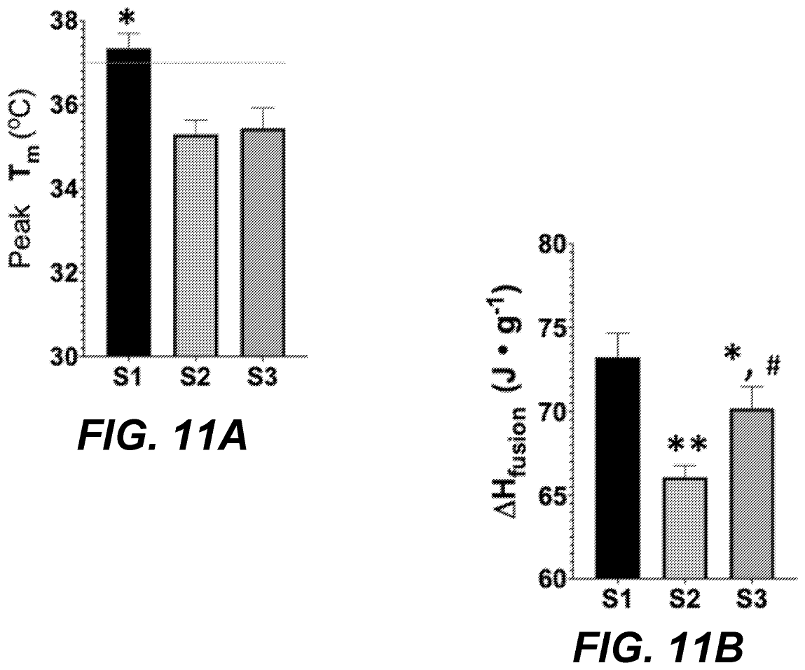

FTIR spectra of partially cured PGD can reveal a larger —OH stretch (FIG. 9) and decreased symmetric and asymmetric —CH₂ stretches. Without wishing to be bound by any particular scientific theory, the stretches can indicate further progression of the polycondensation reaction leading to further polymerization and crosslinking of the S2 and S3 polymers. Increased ester, alkoxy, and ether stretches (FIG. 9) in S2 and S3 polymers compared to S1 can also indicate greater crosslinking and polymerization, without wishing to be bound by any particular scientific theory. The carbonyl stretch (FIG. 9) can reveal no significant difference since this group is largely conserved in both the precursor and the polymer. FTIR spectra of S2 and S3 samples can reveal no significant differences indicating that the one step and multi-step curing resulted in similar molecular architecture. Similarly, the melt transition of S1 can indicate a heterogeneous polymer network with two characteristic melt transitions, whereas the fully cured S2 and S3 polymers can exhibit a single melt transition (FIG. 10). The increased crosslink density within S2 and S3 polymers also can result in a significant downshift (FIG. 11A, p<0.01) of the peak melt temperature (Tₘ), which for a semi-crystalline chemically crosslinked polymer can be indicative of the shape memory transition temperature (Tₜᵣₐₙₛ) which is a critical parameter for delivery and deployment in vivo.

Increased enthalpy of fusion can suggest increased relative crystallinity of S1 compared to S2 (FIG. 11B, **p<0.0001) and S3 (FIG. 11B, *p<0.01). Without wishing to be bound by any particular scientific theory, with greater crosslinking, there is lower chain mobility constraining the arrangement of polymer chains into crystalline lamella which are in line with these results. Interestingly, the S3 polymer can exhibit greater enthalpy of fusion (FIG. 11B, #p<0.002) suggesting greater crystallinity relative to S2 polymer. This suggests that S3 polymer can be less chemically crosslinked than S2 polymer but has a similar transition temperature to S2 polymer indicating an additional mechanism for forming netpoints.

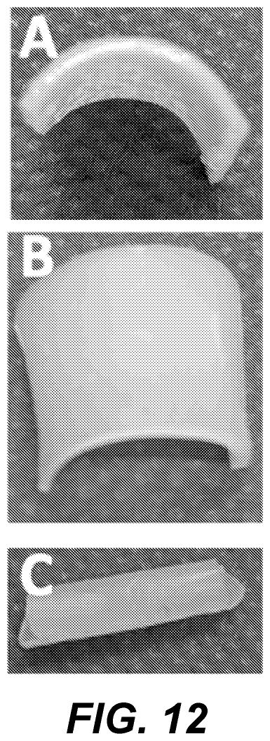

Considering an opening angle of 90° for the deployed patch, 10 mm can be much larger than required for deployment in the right or left pulmonary artery. In accordance with the computational models, a device apposing to 75% of an artery wall having dimensions of 10-20 mm can be considered for deployment into the right or left arterial branches. A rectangular PGD patch without any curvature having dimensions 10 mm CL×20 mm H×500 μm wt can be programmed into a rolled sheet (FIG. 12) and loaded into a catheter sheath. Upon cooling, it can maintain 100% fixity. When deployed in an 11Fr catheter sheath it can achieve 100% recovery. The test patch can recover completely and can be successfully delivered within printed pulmonary artery models.

To resist deformation during delivery, thicker PGD patches can be designed, fabricated, and tested. PGD patches having dimensions (6-25 mm CL)×20 mm H×(0.5-1 mm) wt can be tested in 8-11Fr. A curved design can be explored to improve apposition to the artery wall and surface patterning was considered as design features to reduce dislodgement and improve endothelial alignment and ingrowth. For the second design version, PGD patches having dimensions (16, 14, 12, 9 mm CL)×20 mm H×(0.8 mm) wt×(4 mm, 5 mm, 6 mm ROC) having 0.4×0.4×0.1 mm patterns can be tested in 10-11Fr delivery sheaths. There can be 100% apposition to the tube wall and no dislodgement with both smooth and patterned designs. Opening angles measured after deployment corresponded to expected values. In addition to verification of deployment and recovery, patches can be finally loaded into a 3D printed pulmonary branch model derived from a patient specific pulmonary model. Apposition to the vascular wall and dislodgement from flow can be evaluated.

Pulmonary tissue during gross dissection can be normal and reflected no signs of necrosis. After 3 months in vivo, all samples can be endothelialized, with ⅝ samples exhibiting 100% endothelialization, 2/8 samples exhibiting 85-95% endothelialization, and ⅛ samples exhibiting limited <20% endothelial coverage. Of the endothelialized samples, ⅝ can be fully covered by a neointimal layer. Two of the implants can be partially endothelialized exhibiting 85-95% coverage. On palpation, the integrity of the implants seemed preserved.

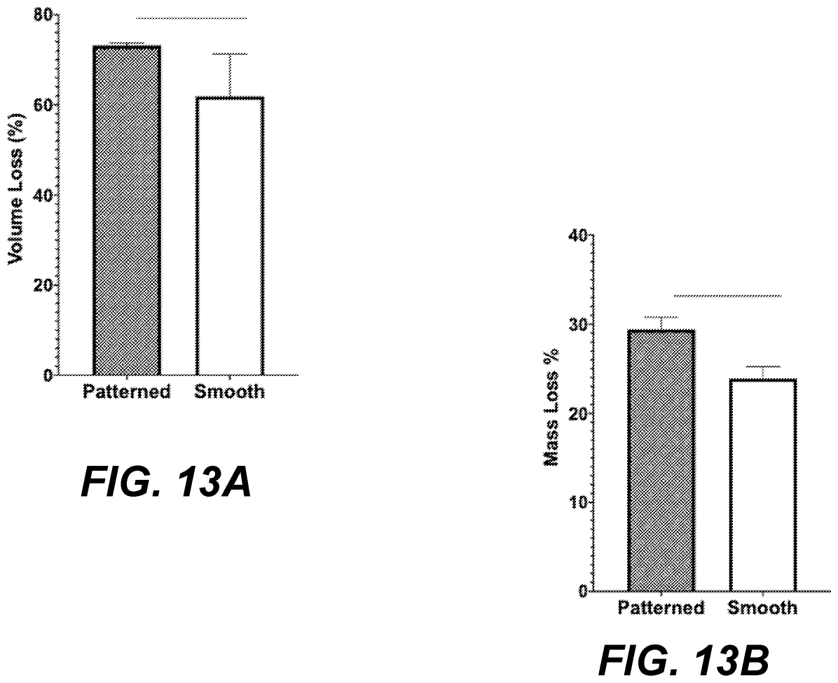

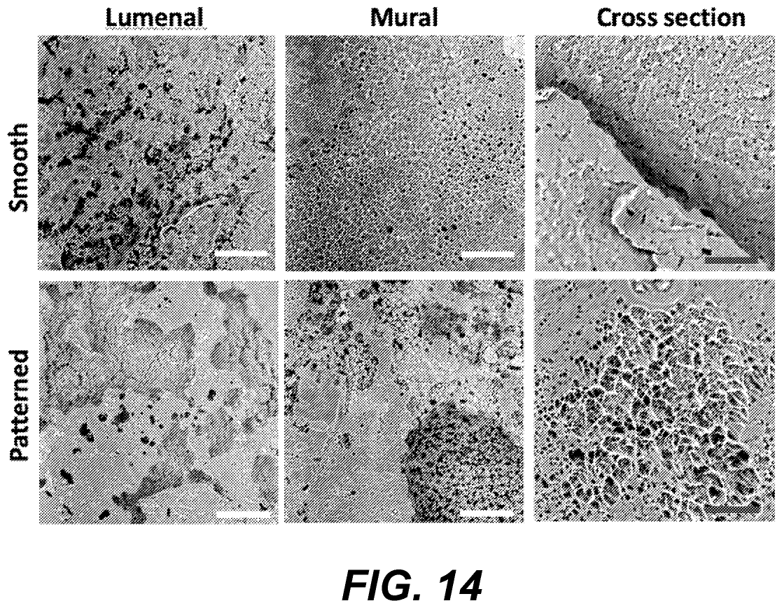

Upon dissection, and clearing, the implants can exhibit clear signs of degradation and tissue ingrowth. Compared to pre-implantation there can be a clear decrease in size of the implants and high-resolution optical microscopy revealed tissue ingrowth on both surfaces with greater growth on the endolumenal surface. Increased degradation can be evident on patterned implants compared to smooth implants. This can be further supported by measured volume loss on patterned compared to smooth implants (FIG. 13A, p<0.01). There can also be a greater mass loss % on patterned implants vs. smooth implants (FIG. 13B, p<0.01). The discrepancy between volume loss and mass loss can be explained by the increase in density of the degraded implants. Without wishing to be bound by any particular scientific theory, the increased density could arise from a greater amount of organic content packed within the implant due to tissue ingrowth. Implant morphology on the lumenal, mural and cross-sectional surfaces reveal surface erosion (FIG. 14). Features can indicate tissue infiltration and growth into the implant. The cross section of all implants can contain small pits and holes that are absent in the preimplant controls.

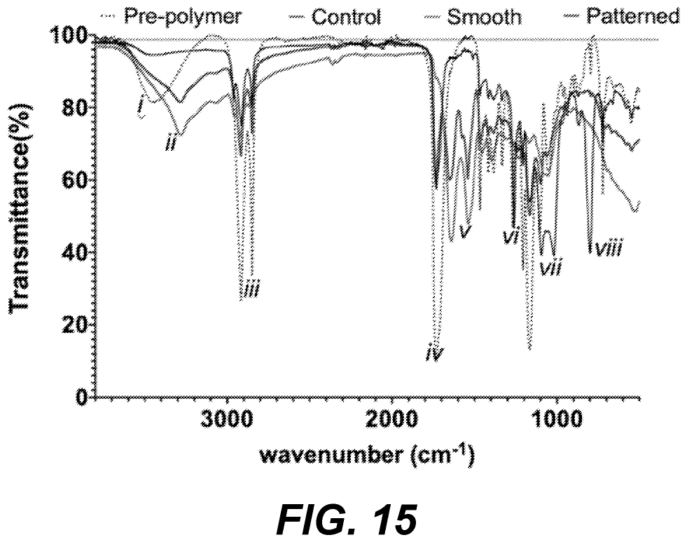

Compositional and thermomechanical evaluation of explanted samples can confirm tissue ingrowth within the polymer and degradation of crosslinks. FTIR spectra of explanted PGD specimens can contain organic components within the polymer confirming tissue ingrowth (FIG. 15). All polymers can contain a characteristic —CH symmetric and asymmetric stretch and a carbonyl stretch. FTIR spectra of PGD pre-polymer can contain an OH stretch indicating significant unreacted glycerol precursors compared to preimplantation controls. This OH stretch can be less evident in the smooth and patterned explants where it could be buried within the shoulder of the amine stretch. Presence of the OH stretch within the explants can suggest degradation and increase in OH groups arising from the organic component and possibly compounded by the degrading polymer backbone. Moreover, presence of the —NH stretch and amide I/II stretch in explanted PGD and an absence in both pre-polymer and pre-implantation controls can confirm the presence of organic components. The CO ester stretch and the CO primary and secondary alcohol stretch in the explants can more closely resemble the uncrosslinked pre-polymer indicating a reduction in crosslinks compared to pre-implantation controls. Moreover, the absence of the characteristic —CH bend can further suggest similarities in polymer structure to uncrosslinked PGD.

In order to successfully functionalize PGD for the implantation in tissues and to promote tissue growth, control of functionalization is essential. The disclosed methods can control how the functionalization occurs over the area, how much of the area is functionalized, and the density of functionalization to determine the optimal settings for tissue growth. The first step in these settings can be the concentration of EDC added post alkylation that can later attach the protein. This example can test varying concentrations of EDC and their ultimate functionalization with FITC-BSA. The functionalization can be performed according to any and/or all of the following:
18 3-mm diameter PGD circles;
50 mL of 0.1M NaOH (1.5 mL per trial) (ref 52770 lot RNB61063);
dDi Water;
50 mL of 0.01 M HCI (1.5 mL per trial) (ref. H9892 lot. ANB60669);
50 mL of MES buffer (ref. J60763 lot X10D502);
10 g of EDC (ref. 22980 lot 51254719); and
5.75 g of NHS (ref. 24500 lot. SR260580).
Calculations can be performed according to any and/or all of the following:
Molecular weight:
EDC: 191.70 g/mol;
NHS: 115.09 g/mol;
FITC-BSA: 389.4 g/mol; and
Volume Calculations:

$$\\ N H S and E D C solution = \frac{1.5 mL}{1 sample} * 9 samples = 13.5 mL + 10 mL ( extra ) = 25 mL$$

$$\\ B C A - Assay solution = \frac{100 µL}{sample} * 81 samples = 8.1 mL + extra = 15 mL$$

$$\\ F I T C - B S A solution = \frac{100 µL}{sample} * 15 samples + 1000 µL = 2.5 mL + extra = 5 mL .$$

25 mL of 1 M solution for EDC and NHS:

$$\\ N H S = \frac{115.09 g}{1 mol} * \frac{1 mol}{1 L} * \frac{1 L}{1000 mL} * 25 mL = 2.87725$$

$$\\ E D C = \frac{191.7 g}{1 mol} * \frac{1 mol}{1 L} * \frac{1 L}{1000 mL} * 25 mL = 4.7925 .$$

FITC-BSA×mL of 1 mM solution×X mL:

$$\\ F I T C - B S A = \frac{389.4 g}{1 mol} * \frac{1 mol}{1000 mM} * \frac{1 mM}{1 L} * \frac{1 L}{1000 mL} * 10 mL = 3.894 mg .$$

While the present disclosure has been described in connection with a plurality of exemplary aspects, as illustrated in the various figures and discussed above, it is understood that other similar aspects can be used, or modifications and additions can be made to the described aspects for performing the same function of the present disclosure without deviating therefrom. For example, in various aspects of the disclosure, methods and compositions were described according to aspects of the presently disclosed subject matter. However, other equivalent methods or composition to these described aspects are also contemplated by the teachings herein. Therefore, the present disclosure should not be limited to any single aspect, but rather construed in breadth and scope in accordance with the appended claims.

1. An implant material comprising:
a shape memory polymer comprising:
at least one monomer unit of glycerol and at least one monomer unit of dodecanedioate; and
a functionalized surface.

2. The implant material of claim 1, wherein the shape memory polymer has a melt transition temperature from approximately 25° C. to approximately 45° C.

3. The implant material of claim 1, wherein the shape memory polymer takes a first shape at a first environmental temperature;
wherein the shape memory polymer takes a second shape at a second environmental temperature;
wherein the second environmental temperature is greater than the first environmental temperature; and
wherein a melt transition temperature of the shape memory polymer is greater than or equal to the first environmental temperature and less than the second environmental temperature.

4. The implant material of claim 2, wherein the shape memory polymer is an elastomer above the melt transition temperature and a thermoplastic below the melt transition temperature.

5.-6. (canceled)

7. The implant material of claim 1, wherein the functionalized surface comprises a plurality of laser cut suture holes.

8. The implant material of claim 1, wherein the functionalized surface comprises at least one functional group.

9. The implant material of claim 8, wherein at least one functional group comprises a bioactive agent.

10. The implant material of claim 1, wherein the molar ratio of the at least one monomer unit of glycerol to the at least one monomer unit of dodecanedioate is from approximately 10:1 to approximately 1:10.

11. The implant material of claim 1, wherein the implant material has a biodegradation time when implanted in vivo from approximately 4 months to approximately 24 months.

12. A method comprising:
forming the implant material of claim 1 into one or more of an implant, a patterned mesh, or a molded medical device.

13. The method of claim 12, wherein the implant is in a form selected from a group consisting of a sheet, a membrane, a mesh, a sponge, a patch, a molded medical device, and a combination thereof.

14. A method comprising:
depositing a shape memory polymer resin into a mold, the shape polymer resin comprising at least one monomer unit of glycerol and at least one monomer unit of dodecanedioate;
partially curing the shape memory polymer resin to form a shape memory elastomer;
cutting the shape memory elastomer; and
curing the shape memory elastomer to form an implant material;
wherein the shape memory elastomer takes a first shape at a first environmental temperature and a second shape at a second environmental temperature;
wherein the second environmental temperature is greater than the first environmental temperature; and
wherein the shape memory elastomer has a melt transition temperature greater or equal to the first environmental temperature and less than the second environmental temperature.

15. The method of claim 14, wherein one or more of:
the mold comprises one or more patterns having features of at least approximately 5 microns or greater;
the mold has a surface having a treatment thereon;
the mold has a surface with a nonfouling release agent;
the cutting comprises laser cutting the shape memory elastomer into a form factor;
the melt transition temperature is from approximately 25° C. to approximately 45° C.;
the shape memory elastomer is an elastomer in the first shape and a thermoplastic in the second shape;
the molar ratio of the at least one monomer unit of glycerol to the at least one monomer unit of dodecanedioate is from approximately 10:1 to approximately 1:10; or
the implant material has a biodegradation time when implanted in vivo from approximately 4 months to approximately 24 months.

16-17. (canceled)

18. The method of claim 15, wherein the form factor is selected from a group consisting of a sheet, a membrane, a mesh, a sponge, a patch, a molded medical device, and a combination thereof.

19. (canceled)

20. The method of claim 14, wherein the melt transition temperature is from approximately 31° C. to 35° C.

21-24. (canceled)

25. The method of claim 14 further comprising laser cutting into a functionalized surface of the shape memory elastomer a plurality of suture holes.

26. (canceled)

27. The method of claim 14, wherein the shape memory elastomer comprises a functionalized surface comprising a bioactive agent.

28-29. (canceled)

30. The implant material made according to the method of any of claims claim 14.

31. The implant material of claim 1, wherein one or more of:
the shape memory polymer has a first shape, a second shape, and a melt transition temperature;
the molar ratio of the at least one monomer unit of glycerol to the at least one monomer unit of dodecanedioate is from approximately 10:1 to approximately 1:10;
the shape memory polymer takes the first shape at a first environmental temperature and the second shape at a second environmental temperature, the second environmental temperature being greater than the first environmental temperature;
the melt transition temperature is greater than or equal to the first environmental temperature and less than the second environmental temperature;
the shape memory polymer is an elastomer above the melt transition temperature and a thermoplastic below the melt transition temperature;
the first shape is selected from a group consisting of a curved shape and a tubular shape; or
the second shape is a tubular shape comprising a cut along a longitudinal axis of the implant material;
